(19) United States
(12) Patent Application Publication
(10) Pub. No.: US 2015/0118760 A1
(43) Pub. Date: Apr. 30, 2015

(54) FLUORESCENT SENSING OF VAPORS USING TUBULAR NANOFIBRIL MATERIALS

(71) Applicants: Ling Zang, Salt Lake City, UT (US); Yanke Che, Salt Lake City, UT (US); Helin Huang, Salt Lake City, UT (US)

(72) Inventors: Ling Zang, Salt Lake City, UT (US); Yanke Che, Salt Lake City, UT (US); Helin Huang, Salt Lake City, UT (US)

(73) Assignee: University of Utah Research Foundation

(21) Appl. No.: 14/237,770

(22) PCT Filed: Aug. 8, 2012
(86) PCT No.: PCT/US2012/049998

§ 371 Date: Oct. 14, 2014

Related U.S. Application Data
(60) Provisional application No. 61/574,738, filed on Aug. 8, 2011.

(51) Int. Cl. G01N 33/00; G01N 21/64

(52) U.S. Cl. CPC G01N 33/0057; G01N 21/6408
USPC 436/110; 422/82.08

(57) ABSTRACT

A fluorescence-based sensor can comprise a nanofiber mass of nanofibers having tubular morphology and a fluorescence detector, where fluorescence of the nanofibers decreases upon contact with a nitro-containing compound. The nanofibers can comprise carbazole-cornered, arylene-ethynylene tetracyclic macromolecules of formula I: where R1-R4 are alkyl-containing groups. The tubular morphology allows for highly selective detection of trinitrotoluene over other nitro-based compounds and oxidizing organic compounds.

### RELATED APPLICATION

This application claims the benefit of copending U.S. Provisional Patent Application Ser. No. 61/574,738, filed on Aug. 8, 2011, which is hereby incorporated by reference in its entirety.

### GOVERNMENT INTEREST

This invention was made with government support under Grants CHE0931466 awarded by the National Science Foundation and Department of Homeland Security Grant 2009-ST-108-LR0005. The Government has certain rights in this invention.

### FIELD OF THE INVENTION

This invention relates generally to materials and chemical sensors. Therefore, the present invention relates generally to the fields of chemistry and materials science.

### BACKGROUND

Various vapor sensing devices have been employed to provide a means for monitoring and controlling organic compounds. Such devices can include chemiresistors and semiconductors. Compared to the inorganic chemiresistors, organic semiconductors can offer facile deposition procedure as well as various choices and easy tuning of bind receptors for analyte molecules. Although organic field-effect transistors (FETs) can also be used as chemiresistors, the fabrication is relatively complicated and the performance is affected by many factors, like boundary grain, surface morphology, molecular structure, etc.

Trace explosive detection has drawn intense attention due to the increasing concern in homeland security, military operation safety, and environmental and industrial safety control. Among the current detection technologies, fluorescence sensors represent one class of detection modalities which offer some benefits. Fluorescence sensing is suited for vapor detection, such as screening and monitoring explosive threats hidden deeply in passenger luggage or commercial cargo containers. However, fluorescence sensory materials (e.g. polymers) tend to respond broadly to a class of analytes, rather than a single specific target. For example, most of the sensory materials developed for the detection of TNT are also sensitive to other nitro-organic compounds and many other oxidizing reagents such as quinones. The lack of specific chemical selectivity limits their effective deployment as a useful sensor. As such, novel sensor devices which are more selective continue to be sought through ongoing development and research efforts.

### SUMMARY

It has been recognized that it would be advantageous to develop a chemical sensor compound for selective detection of specific explosive materials. As such, in one embodiment, a fluorescence-based sensor can comprise a nanofiber mass of nanofibers having a tubular morphology and a fluorescence detector, where fluorescence of the nanofibers decreases upon contact with a nitro-containing compound. The nanofibers can comprise carbazole-cornered, arylene-ethynylene tetracyclic macromolecules of formula I:

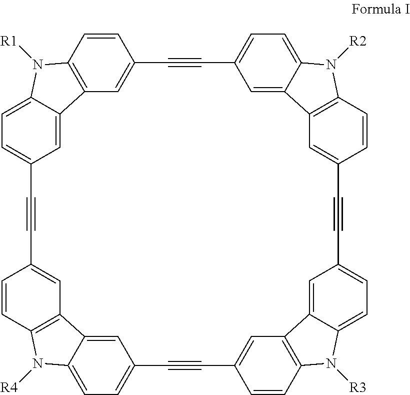

where R1-R4 are side-chain groups and wherein the macromolecules are cofacially stacked.

Additionally, a method of manufacturing the nanofibers can comprise solvating the carbazole-cornered, arylene-ethynylene tetracyclic macromolecules in a first organic solvent forming a macromolecule solution, admixing the macromolecule solution with a second organic solvent forming a binary solvent system, and cooling the binary solvent system to a temperature of at least 4° C. for a period of at least 6 days thereby forming the nanofibers having tubular morphology.

Further, a method of detecting explosives can comprise exposing nanofibers having a tubular morphology to a target sample, the nanofibers comprising any of the macromolecules discussed herein, and measuring fluorescence responses of the nanofibers.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIGS. 1A-B are SEM images of entangled alkyl-substituted, carbazole-cornered, arylene-ethynylene tetracyclic macromolecule nanofibers deposited on glass (A) where R1-4 are C10 linear alkyl and (B) where R1-4 are C14 linear alkyl in accordance with one embodiment of the present invention;

FIGS. 1C-D are SEM images of entangled non-tubular nanofibers from carbazole ethynylene oligomers;

FIG. 1E is an enhanced schematic illustration showing tubular or nanoporous packing and rectangular lattice of a nanofiber in accordance with one embodiment of the present invention;

FIGS. 2A-B are TEM images of nanoporous nanofibers of the present invention in accordance with one embodiment of the present invention;

FIGS. 3A-B are X-ray diffraction patterns of nanoporous nanofibers of the present invention in accordance with one embodiment of the present invention

FIG. 4 is the absorption (left) and fluorescence (right) spectra of nanoporous nanofibers (solid) and individual molecule dissolved in chloroform solution (dashed) in accordance with one embodiment of the present invention;

FIG. 5 is shows X-ray diffraction patterns of solid nanofibers;

FIGS. 6 A-B are (a) the absorption (left) and fluorescence (right) spectra of solid nanofibers (solid) and individual molecules dissolved in chloroform solution (dashed); (b) the absorption (left) and fluorescence (right) spectra of solid nanofibers (solid) and individual molecules dissolved in chloroform solution (dashed);

FIG. 7A is a fluorescence spectrum of nanofibers of the present invention before (upper) and after (lower) exposure to saturated TNT vapor (5 ppb) for 1 minute in accordance with one embodiment of the present invention;

FIG. 7B shows post-exposure fluorescence intensity change (I/Io) measured over the nanofibers of 2A as function of time after reopen to clean air; the values at time zero represent the fluorescence intensity measured after 20 s of exposure to the saturated vapor of various oxidizing reagents and 1 min of exposure to the saturated vapor of TNT in accordance with one embodiment of the present invention;

FIG. 7C shows post-exposure fluorescence intensity change (I/Io) measured over three different nanofibers as function of time after reopen to clean air; the values at time zero represent the fluorescence intensity measured after 20 s of exposure to the saturated vapor of DNT (100 ppb) in accordance with one embodiment of the present invention;

FIG. 8 shows photocurrents of nanoporous nanofibers of the present invention measured under ambient condition (upper) and under argon condition (lower) in accordance with one embodiment of the present invention;

FIG. 9 is a TEM image of nanoporous nanofibers in accordance with one embodiment of the present invention;

FIG. 10 provides X-ray diffraction patterns of nanoporous nanofibers in accordance with one embodiment of the present invention;

FIG. 11 shows the absorption (left) and fluorescence (right) spectra of nanoporous nanofibers of the present invention (solid) and individual molecules dissolved in chloroform solution (dashed);

FIG. 12A is a fluorescence spectra recorded over nanofibers of the present invention before and after exposure to the saturated TNT vapor (5 ppb) for 10 s in accordance with one embodiment of the present invention;

FIG. 12B is a fluorescence spectra recorded continuously over the nanofibers of 12A at different time intervals after reopen to clean air: 0, 1, 2, 3, 7, 10 min in accordance with one embodiment of the present invention;

FIG. 12C shows post-exposure fluorescence intensity change (I/Io) measured over the nanofibers of 12A as function of time after reopen to clean air; the values at time zero represent the fluorescence intensity measured after 10 s of exposure to the saturated vapor of different oxidizing reagents including TNT in accordance with one embodiment of the present invention;

FIG. 13 shows the relative fluorescence intensity of various nanofibers after exposure to explosive vapors (left) and after recovery by saturated hydrazine vapor (right): (1), porous nanofiber film from compound 4 of scheme 2; (2), porous nanofiber film from compound 5 from scheme 2; (3), spin-cast nanofibril film of compound 3 from scheme 2; (4), nanoporous nanofibers from compound 3 from scheme 2. The porous nanofibers films from compounds 4 and 5 were exposed to DNT saturated vapor for 20 seconds while spin-cast nanofibril film of compound 3 and nanoporous nanofibers from compound 3 were exposed to TNT saturated vapor for 10 seconds (nanoporous nanofibers from compound 3 were kept in dark for another 10 min) before the recovery experiments, where they were immersed in the saturated vapor of hydrazine for 1 min followed by drying in a stream of nitrogen;

FIG. 14 shows post-exposure (initial exposure to the saturated vapor of different oxidizing reagents for 10 seconds) fluorescence intensity change after reopen to clean air for 10 min for various reagents in accordance with one embodiment of the present invention;

FIG. 15 shows relative fluorescence intensity measured as a function of time of vapor exposure for the nanofibers from 12A; four vapor concentration levels: 1, 2-8 ppb; 2, 0.2-1.5 ppb; 3, 50-100 ppt; 4, 20-50 ppt are shown in accordance with one embodiment of the present invention;

FIG. 16 is shows fluorescence quenching efficiency (1−I/I0) as a function of the vapor pressure of TNT: data (error±5%) fitted with the Langmuir equation in accordance with one embodiment of the present invention; and

FIG. 17 shows the relative fluorescence intensity measured as a function of time of exposure to the vapor of RDX produced at 60° C. (the vapor pressure of RDX maintained in the optical chamber was in the range of 0.05-3.2 ppb; the large range of vapor pressure represents the gradient within the chamber from the vapor-out to the vapor-in end).

These figures are not necessarily to scale and actual dimensions may, and likely will, deviate from those represented. Thus, the drawings should be considered illustrative of various aspects of the invention while not being limiting. Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENTS

Before the present invention is disclosed and described, it is to be understood that this disclosure is not limited to the particular process steps and materials disclosed herein because such process steps and materials may vary somewhat. It is also to be understood that the terminology used herein is used for the purpose of describing particular embodiments only. The terms are not intended to be limiting because the scope of the present disclosure is intended to be limited only by the appended claims and equivalents thereof.

It must be noted that, as used in this specification and the appended claims, the singular forms “a,” “an,” and “the” include plural referents unless the context clearly dictates otherwise.

As used herein, “alkyl” refers to a branched, unbranched, or cyclic saturated hydrocarbon group, which typically, although not necessarily, contains from 1 to about 50 carbon atoms, or 1 to about 40 carbon atoms, or 1 to about 30 carbon atoms for example. Alkyls include, but are not limited to, methyl, ethyl, n-propyl, isopropyl, n-butyl, isobutyl, t-butyl, octyl, and decyl, for example, as well as cycloalkyl groups such as cyclopentyl, and cyclohexyl, for example. As used herein, “substituted alkyl” refers to an alkyl substituted with one or more substituent groups. The term “functionalized alkyl” refers to alkyls having functional groups including, but not limited to, heteroatoms, e.g., oxygen and nitrogen; carbonyls; aromatic groups; multiple bonds; and ionic groups. Such functionalized alkyls can therefore include esters, ethers, aryls, ketones, aldehydes, carboxylic acids, alcohols, amines, amides, salts, etc. The term “heteroalkyl” refers to an alkyl in which at least one carbon atom is replaced with a heteroatom. If not otherwise indicated, the term “alkyl” includes unsubstituted alkyl, substituted alkyl, lower alkyl, functionalized alkyls, and heteroalkyl.

As used herein, “nanofiber” refers to any elongated structure having a nanoscale cross-section such as, but not limited to, nanowires, nanobelts, nanoribbons, or other nanofibrous materials.

As used herein, “carbazole-cornered, arylene-ethynylene tetracyclic macromolecule” refers to molecules having the structure of Formula 1:

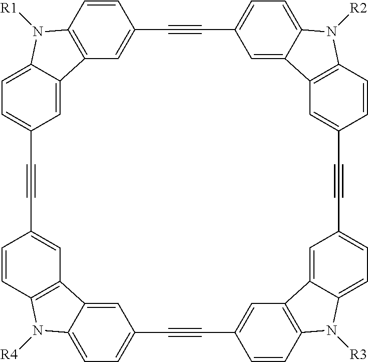

where R1-R4 are side-chain groups which facilitate cofacial stacking to form tubular nanofibers. The side chains can be a variety of groups such as, but not limited to, unsubstituted or substituted, linear or branched, alkyl chains, carboxy groups having alkyl chains, polyethylene glycols, polyethers, aromatic having alkyl side chains, and the like. Alkyl chains include non-functionalized alkyl chains, e.g., linear C₁₄ chain, and functionalized alkyl chains, e.g. (C═O)OC(CH₃)₂(CH₂)₁₁CH₃.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 wt % to about 5 wt %” should be interpreted to include not only the explicitly recited values of about 1 wt % to about 5 wt %, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The present inventors have discovered the effective use of organic nanofibers assembled from alkyl-substituted, carbazole-cornered, arylene-ethynylene tetracyclic macromolecules for detection of nitro-containing compounds using fluorescence detection. Additionally, the present inventors have discovered a novel manufacturing process of producing nanofibers that provide tubular morphology. Tubular nanofibers exhibit a unique morphology where an interior channel is formed by cofacially stacked cyclic molecules which align along a substantial or entire length of the nanofiber. This interior channel allows for a highly specific detection of trinitrotoluene which diffuses into the interior channel to affect fluorescence as described in more detail herein. Such novel structure of the nanofibers can provide selective detection of certain nitro-containing compounds, including trinitrotoluene (TNT).

Specifically, in one embodiment, a fluorescence-based sensor can comprise a nanofiber mass of nanofibers having tubular morphology and a fluorescence detector, where fluorescence of the nanofibers decreases upon contact with a nitro-containing compound. In one embodiment, the fluorescence detector can be a fluorimeter.

The nanofibers can comprise carbazole-cornered, arylene-ethynylene tetracyclic macromolecules of formula I:

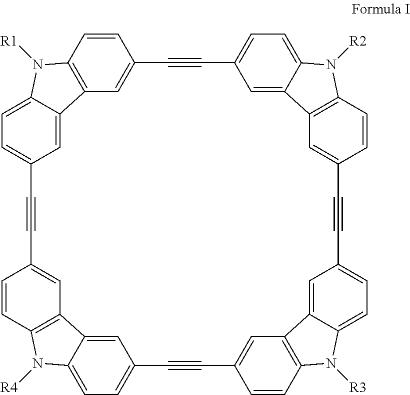

where R1-R4 are side-chain groups and wherein the macromolecules are cofacially stacked to form a tubular morphology. The side chains can be an alkyl-containing group such as, but not limited to, unsubstituted or substituted, linear or branched, alkyl chains, functionalized alkyls including carboxy groups having alkyl chains, polyethylene glycols, polyethers, aromatic having alkyl side chains, and the like. In one embodiment, R1-R4 can be individually selected from C3 to C18 alkyl chains. In one aspect, R1-R4 can be C14 alkyl chains, and in one specific aspect, can be C14 linear alkyl chains. In another embodiment, R1-R4 can be individually selected from C3 to C18 functionalized alkyl chains. In one aspect, R1-R4 can be (C═O)OC(CH₃)₂(CH₂)₁₁CH₃. Other side-chain groups can include

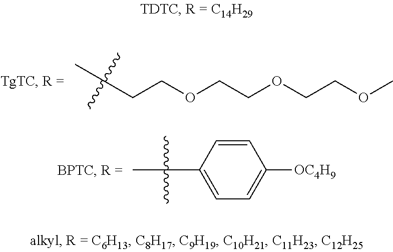

Typically, R1-R4 can be identical groups, although combinations of different groups may be used. The tubular nanofibers can generally have a diameter of about 10 nm to about 100 nm.

Generally the nanofibers are present in the sensor as a nanofiber mass. In one embodiment, the nanofiber mass can be a film. The film can be highly porous. In one embodiment, the film can have a porosity ranging from 10 nm to 2 μm. In another embodiment, the film can have a thickness ranging from 20 nm to 10 μm. The individual tubular nanofibers can have diameters ranging from about 10 nm to about 50 nm, and in some cases from about 20 nm to about 30 nm. The tubular morphology typically involves multiple parallel strands which are each made of the cofacially stacked macromolecules. Although the number of parallel strands can vary, there are often from about 10 to about 200 strands per tubular nanofiber.

The present sensors can detect nitro-containing compounds using fluorescence quenching. Accordingly, in one aspect, the fluorescence can be decreased for at least 5 minutes after exposure to the nitro-containing compound. In another aspect, the fluorescence can be decreased for at least 10 minutes after exposure to the nitro-containing compound. In one embodiment, the nitro-containing compound can adsorb on the nanofiber mass for at least 5 minutes. In one aspect, the nitro-containing compound can adsorb on the nanofiber mass for at least 10 minutes.

The present sensors have been found to have excellent sensitivity. Accordingly, in one embodiment, the sensor can detect the nitro-containing compound in a concentration as low as 1 ppm. In one aspect, the sensor can detect the nitro-containing compound in a concentration as low as 1 ppb. In another aspect, the sensor can detect the nitro-containing compound in a concentration as low as 1 ppt.

In one embodiment, the change in fluorescence can decrease by at least 1% after the nanofiber mass is contacted with the nitro-containing compound. In one aspect, the change in fluorescence can decrease by at least 3% after the nanofiber mass is contacted with the nitro-containing compound. In another aspect, the change in fluorescence can decrease by at least 5% after the nanofiber mass is contacted with the nitro-containing compound.

Without intending to be bound by any particular theory, the present inventors have theorized that the present tubular morphology of the present nanofibers can allow for selective detection of TNT over other oxidizing organic compounds. The selectivity can be attributed to the shape of the interior channel and complimentary size with TNT. Post-exposure diffusion profiles are characteristic of specific analytes and TNT profiles are particularly distinct. Diffusion of TNT from these interior channels can be markedly slower than the nitro-aromatic analogues and other oxidizing organic compounds. Such selectivity can be accomplished by measuring a different post-exposure fluorescence change profile affected by TNT compared to the other oxidizing organic compounds. In one specific aspect, the nitro-containing compound can be trinitrotoluene (TNT). In another specific aspect, the nitro-containing compound can be 1,3,5-Trinitroperhydro-1,3,5-triazine (RDX).

Generally, the present sensor comprises nanofibers having a tubular morphology and a fluorescence detector. Additionally, the sensor can comprise a housing that contains the nanofibers and fluorescence detector. As such, in one embodiment, the sensor can be portable. Further, the sensor can comprise an opening for air intake and can further comprise a viewing area for fluorescence viewing and/or a fluorescence readout. As such, in one embodiment, the fluorescence quenching can be visible or qualitative measurement. Additionally, the fluorescence can be detected via a fluorescence detector and converted to a quantified numerical value which can be transformed to a digital number viewable in the fluorescence readout on the sensor. As such, the sensor can comprise an explosives indicator display based on the fluorescence responses. In one aspect, the explosives indicator can be based on a quantitative measurement. In another aspect, the explosives indicator can be based on a qualitative measurement. Further, the sensor can be configured to move air or gaseous sample through the sensor such that the air or sample contacts the nanofibers within a sensor housing. The sensor housing can include an inlet to allow external air to enter in a passive or forced manner. As such, the sensor can comprise a fan or other mechanical structure that allows for the movement of the air or gaseous sample within the sensor housing.

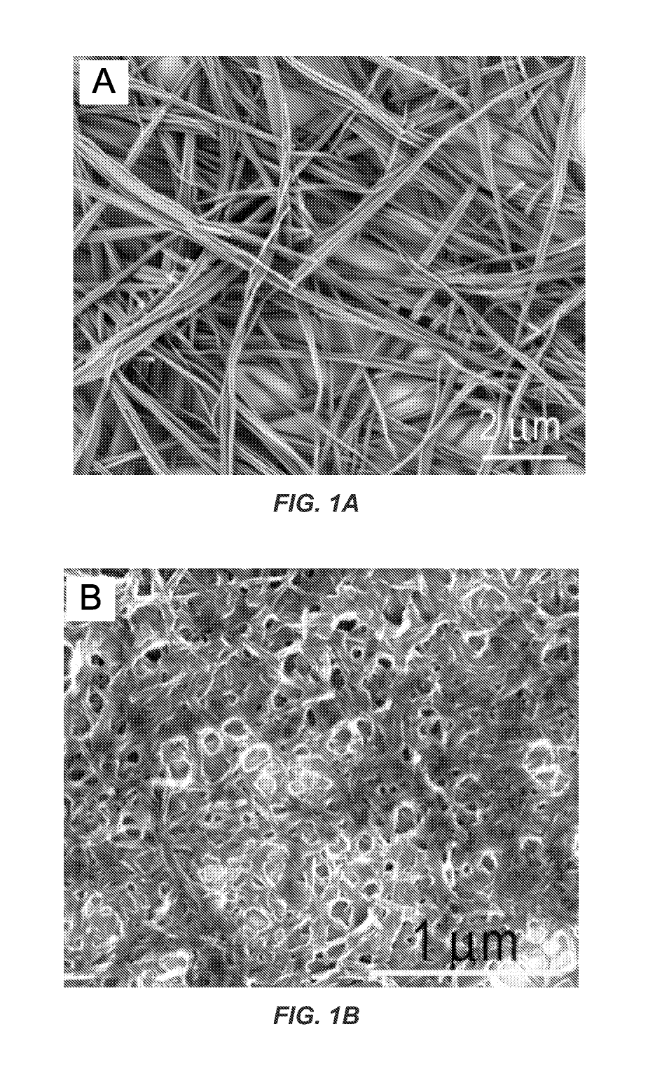

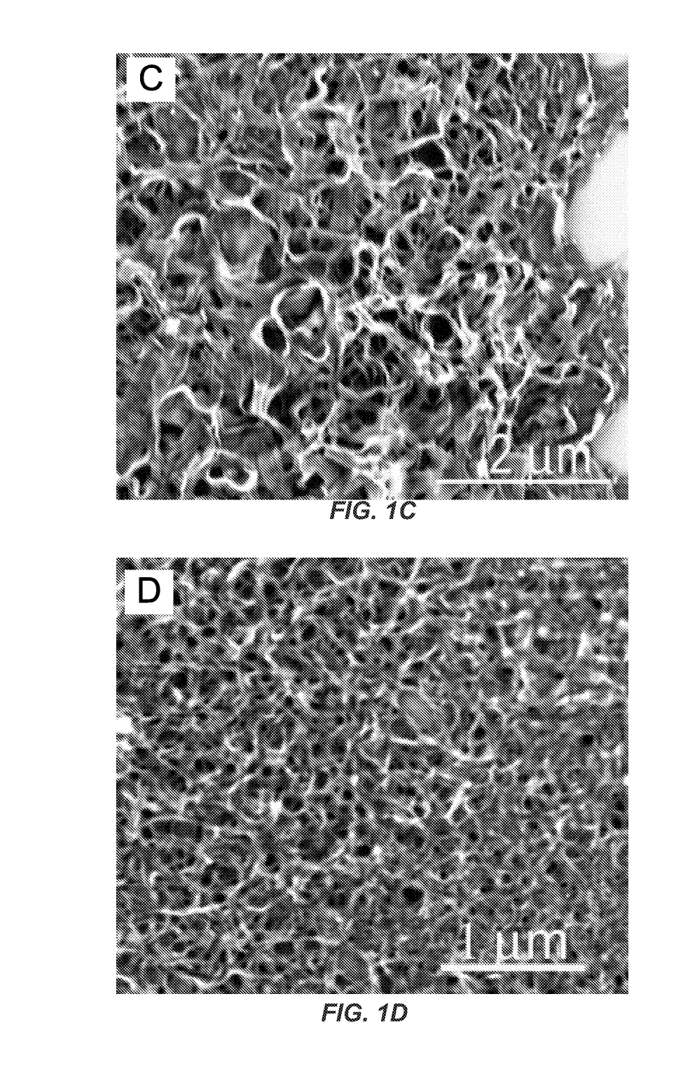

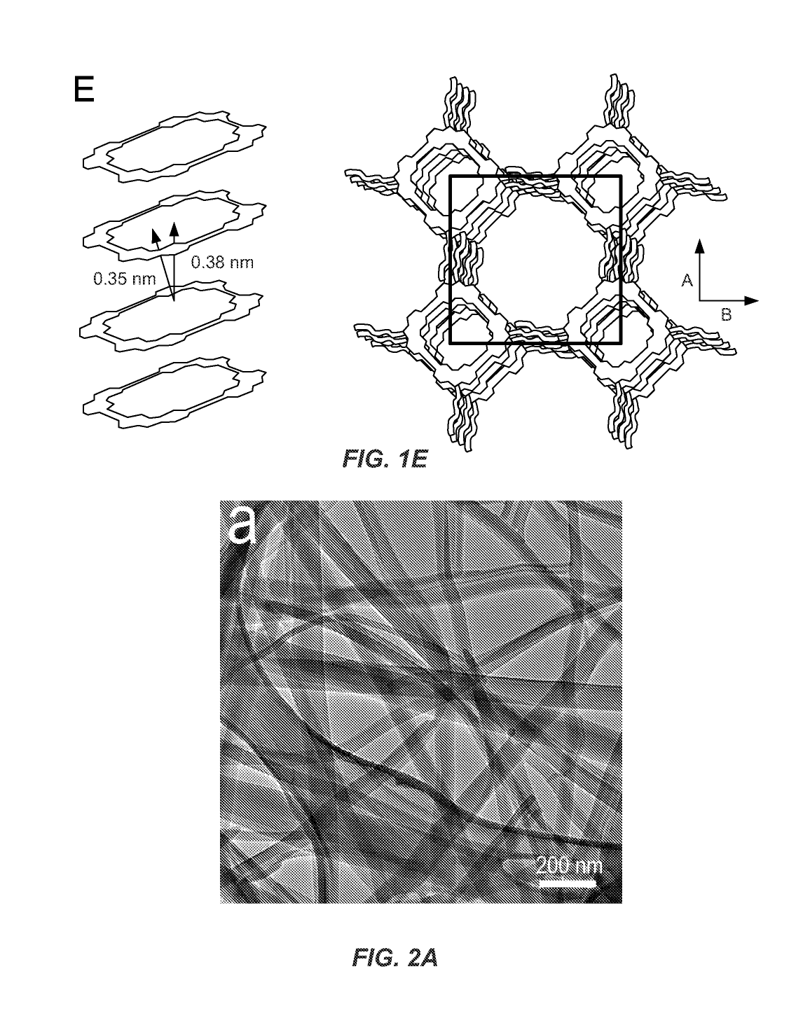

A method of manufacturing the nanofibers can comprise solvating the carbazole-cornered, arylene-ethynylene tetracyclic macromolecules in a first organic solvent forming a macromolecule solution, admixing the macromolecule solution with a second organic solvent forming a binary solvent system, and cooling the binary solvent system to a temperature of at least 4° C. for a period of at least 6 days thereby forming the nanofibers having tubular morphology. The tubular morphology presently achieved is generally a function of temperature and time, where the combination of both variables need to be sufficient such that the macromolecules are able to stack such that the cores of the macromolecules are aligned, e.g., as indicated in FIG. 1E. As discussed herein, in one embodiment, a solution of the macromolecules can be cooled to a temperature of at least 4° C. for a period of at least 6 days thereby forming nanofibers having tubular morphology. In one aspect, the period of time can be at least 7 days.

Generally, the solvents used in the manufacturing process can include any solvents that can form a binary solvent system. In one embodiment, the first organic solvent can be a halogen-containing solvent. In one aspect, the first organic solvent can be chloroform. In another embodiment, the second organic solvent can be an alcohol. In one aspect, the second organic solvent can be ethanol.

The carbazole-cornered, arylene-ethynylene tetracyclic macromolecules can be dissolved in the first organic solvent sufficient to solvate the macromolecules. The concentration of the macromolecule solution can range from 0.01 mM to 10 mM. In one aspect, the concentration of the macromolecule solution can range from 0.05 mM to 0.2 mM.

A method of detecting explosives can comprise exposing nanofibers having a tubular morphology to a target sample, the nanofibers comprising any of the macromolecules discussed herein, and measuring fluorescence responses of the nanofibers. The method can further comprise exposing the nanofibers to an ambient gas and measuring a post-exposure fluorescence. As discussed herein, the post-exposure fluorescence can be used to provide a rate of diffusion of the target compound from the nanofibers allowing for selective detection of specific nitro-containing compounds. As such, in one embodiment, the method can selectively detect TNT over other nitro-containing compounds based on the post-exposure fluorescence.

The ambient gas can be such gas that does not interact with the nanofibers or that is capable of displacing the target compound. As such, in one aspect, the ambient gas can comprise molecules that have no or substantially no quenching effect on the nanofibers. In one embodiment, the ambient gas can be a member selected from the group consisting of air, a noble gas, an inert gas, and mixtures thereof.

The method can further comprising recycling the nanofibers by dissolving the nanofibers in a first organic solvent and extracting the nanofibers with a second organic solvent. In one embodiment, the first organic solvent can be a halogen-containing solvent. In one aspect, the first organic solvent can be chloroform. In another embodiment, the second organic solvent can be an aqueous alcohol mixture. In one aspect, the second organic solvent can be a mixture of ethanol and water.

The present fabricated tubular nanofibers provide unique and advantageous features in comparison to bulk-phase materials or solid nanofibers. These features include more organized crystalline structure, tubular morphology, and a large surface area conducive to surface modification. Moreover, when deposited onto a substrate the large number of entangled nanofibers can form highly porous film, with continuous, size variable open pores that facilitate gas collection and diffusion throughout the whole materials. These combined features give the present tubular nanomaterials strong vapor detection performance via fluorescence detection.

### Examples

### Example 1

### Synthesis and Fabrication of Nanofibers

Nanofibers were fabricated using a novel self-assembly process under low temperature conditions, allowing for slow Oswald ripening in a binary solvent system. With this method, long nanofibril structures were formed as revealed by scanning electron microscopy (SEM) imaging (FIG. 1A-D). Specifically, porous nanofibers of the present invention (FIGS. 1A-B) were fabricated by injecting 0.3 mL chloroform solution of the corresponding tetracycle compound (0.15 mM) into 4 mL ethanol in a test tube followed by 7 days aging in the refrigerator. The nanofibers thus formed were transferred onto glass surface by spin-casting of the suspending solution (2000 r/m) for sensing test.

Additionally, the solid nanofibers made from carbazole oligomers (FIGS. 1C-D) were fabricated by injecting 0.2 mL chloroform solution of corresponding compound (0.5 mM) into 4 mL ethanol in a test tube followed by 6 days aging in the refrigerator. The nanofibers thus formed were transferred onto glass surface by spin-casting (2000 r/m) the suspending solution for sensing test.

Five nanofibers were fabricated having structures as outlined in scheme 1. Compounds 1-3 correspond to nanofibers of the present invention having tubular morphology as compared to compounds 4-5 that correspond to nanofibers having a solid morphology.

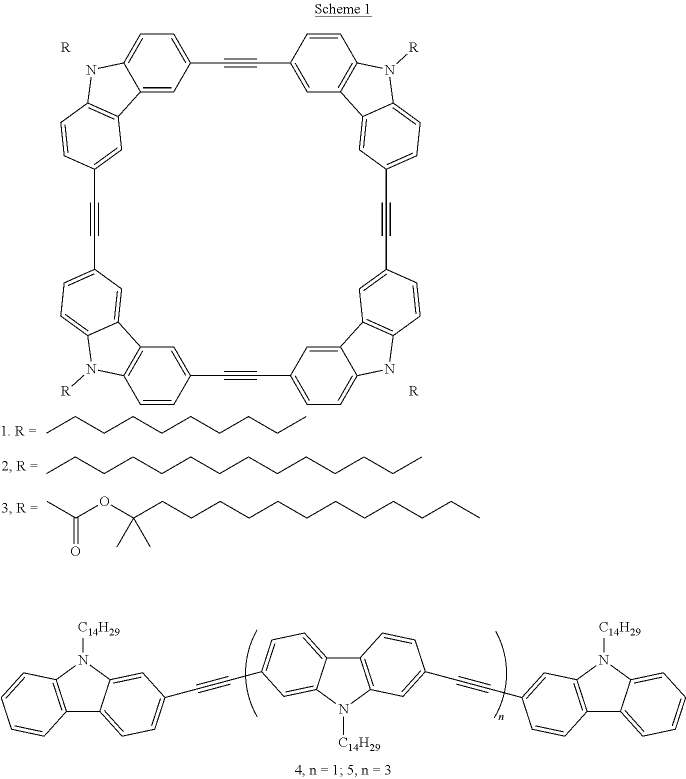

### Example 2

### Structural and Property Characterizations

The following structural properties and characterization were made for compounds 1-5 of Example 1.

Generally, porosity can be achieved by piling (spin-casting) the nanofibers, which were prefabricated in solution (FIG. 1A-D). Among these, well-defined larger nanofibers were fabricated from compound 1, due to its shorter alkyl side chains (FIG. 1A). The large size and straight morphology of the nanofibers make the piling with large pores, or interstices, in the range of microns.

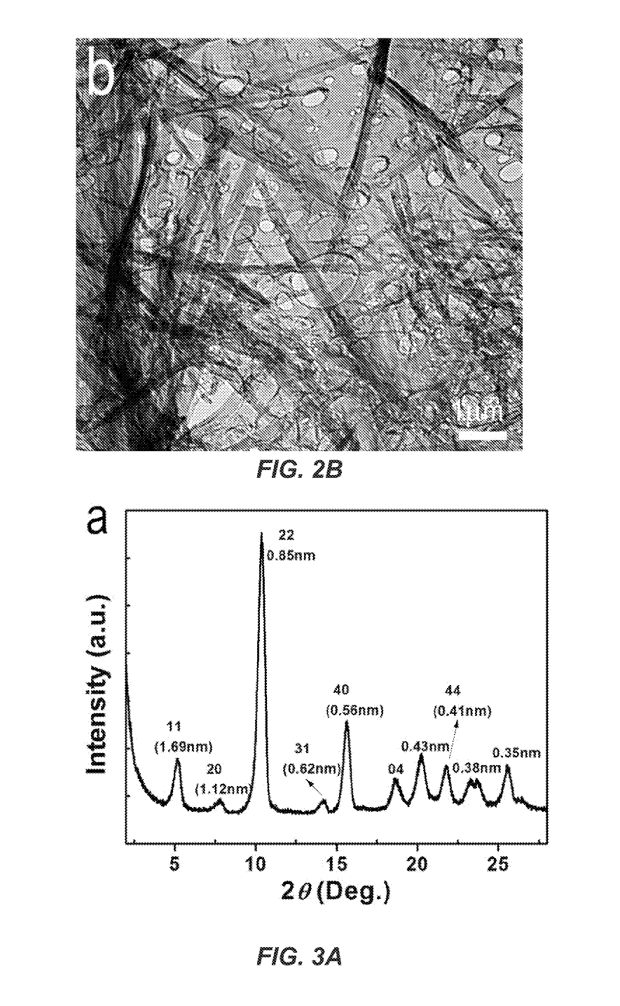

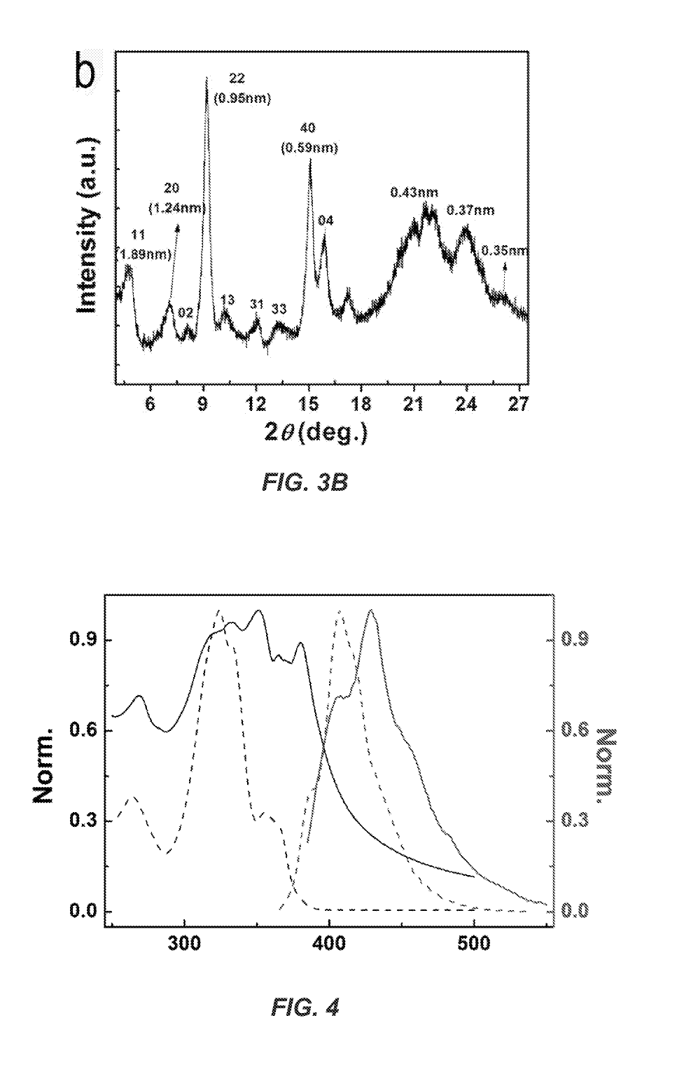

Although the TEM imaging (FIG. 2) does not reveal the tubular structures of nanofibers fabricated from compound 1 or compound 2 (in the similar manner as reported for carbon nanotubes), the long range tubular stacking is evidenced by X-ray diffraction (XRD) measurements (FIG. 3a). XRD of nanofibers of compound 1 displayed pronounced diffraction peaks (FIG. 3), which can be assigned to a two-dimensional (2D) rectangular lattice with lattice parameters a and b of 2.24 nm. The d-spacing values of 0.38 nm at 2θ=23.4° and 0.35 nm at 2θ=25.5°, assignable to the center-to-center distance and π-π stacking distance of tilted macrocycles, respectively, suggesting that molecules of compound 1 most likely adopt a co-facial orientation with a tilting angle of 23° (i.e., small lateral offset of 0.15 nm) relative to the long axis of the resulting column (FIG. 1e).

This slipped stacking of compound 1 is also consistent with the red-shifted absorption and emission as measured over the nanofibers, in comparison to that of individual molecules dissolved in solutions (FIG. 4), indicative of J-aggregate formation. The nanoporous structure as dominated by the tubular stacking was confirmed by the greater intensity observed at higher-order XRD peaks (22, 40), which are typically characteristic of porous tubular nano-structures. The hydrophobic interaction between alkyl chains is also supported by the observed diffraction peak with a d-spacing of ca. 0.43 nm, typical of ordered paraffinic side chains.

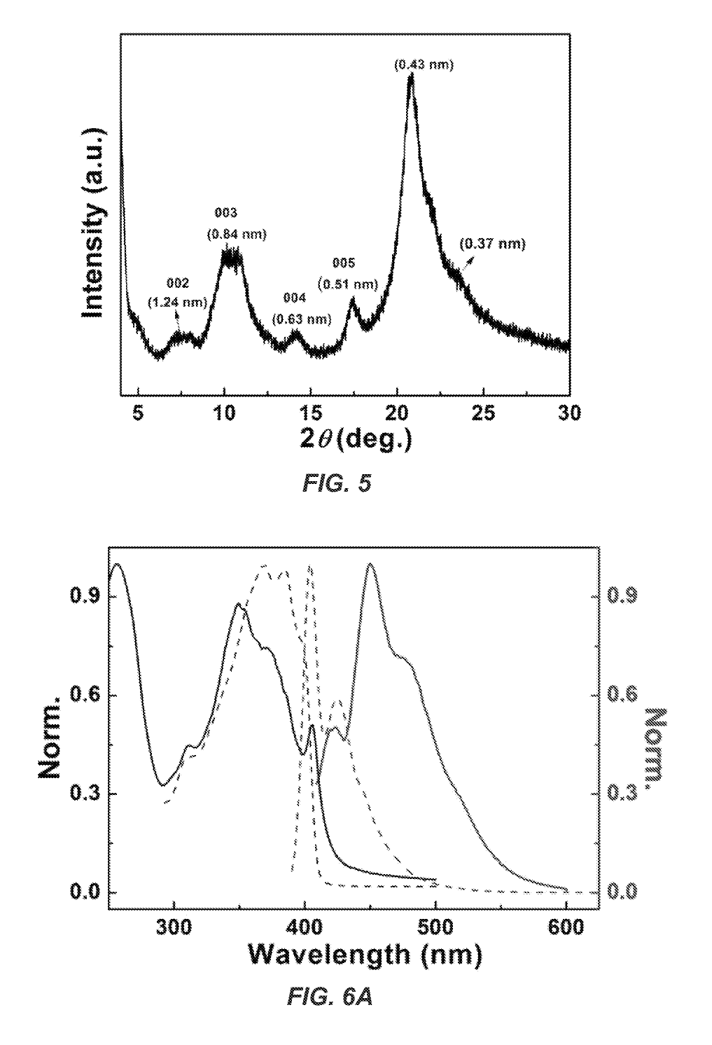

Similarly, the diffraction pattern of compound 2-nanofibers can be indexed to a 2D rectangular lattice but with larger lattice parameters of a and b of 2.48 nm, consistent with longer side chains in compound 2, and higher intensity of the higher-order diffraction peaks (22, 40) indicates the formation of long range order of the porous tubular nanostructure (FIG. 3b). Carbazole-based oligomer (compound 4), lacking a molecular cavity, adopt lamellar stacking into solid nanofibers, as evidenced by its XRD data (FIG. 5), where the peaks can be indexed to higher order diffraction from (002) to (005).

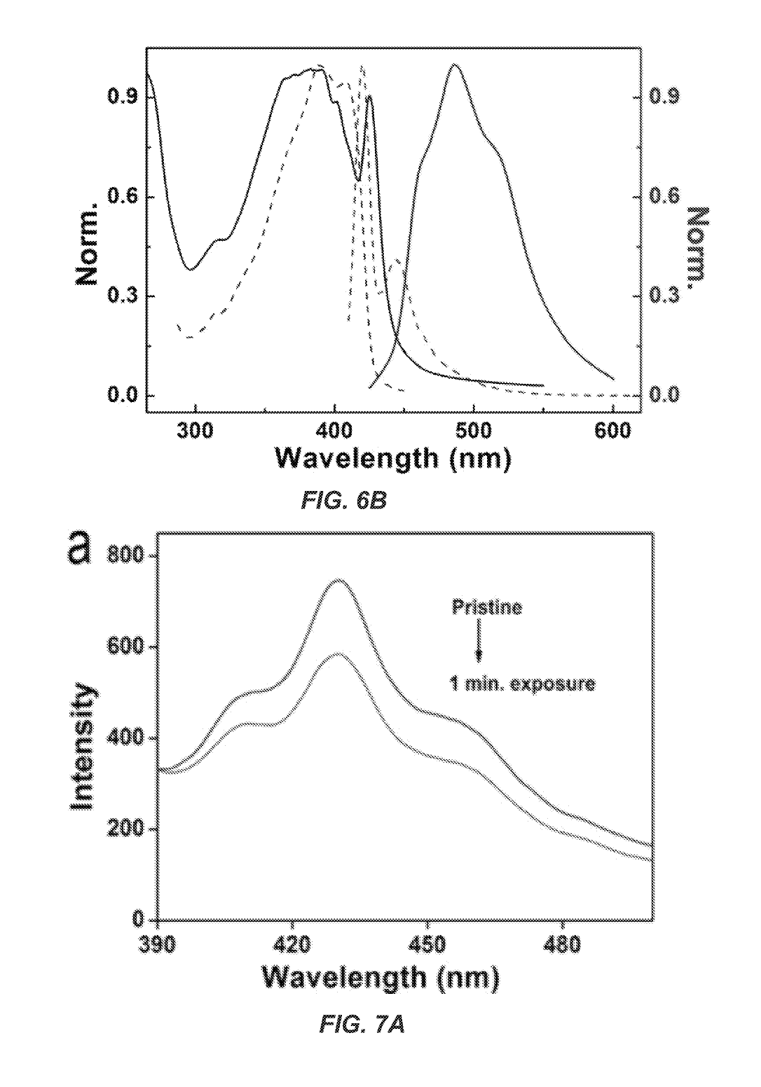

Similar intermolecular stacking is expected for the nanofibers of compound 5 considering the similar absorption and emission spectral change of compound 5 to those of compound 4 (FIG. 6). The above studies show that oligomer molecules can form solid nanofibers and subsequently form exterior porous films upon piling, while tetracycle molecules can form nanoporous nanofibers via tubular stacking which can also possess exterior pores from the piling.

Upon exposure to the oxidizing reagent vapors as those listed in Scheme 2, the emission of nanoporous nanofibers was quenched.

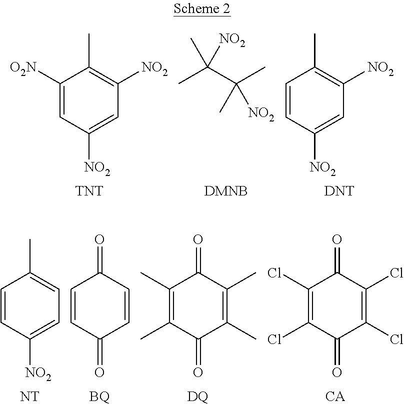

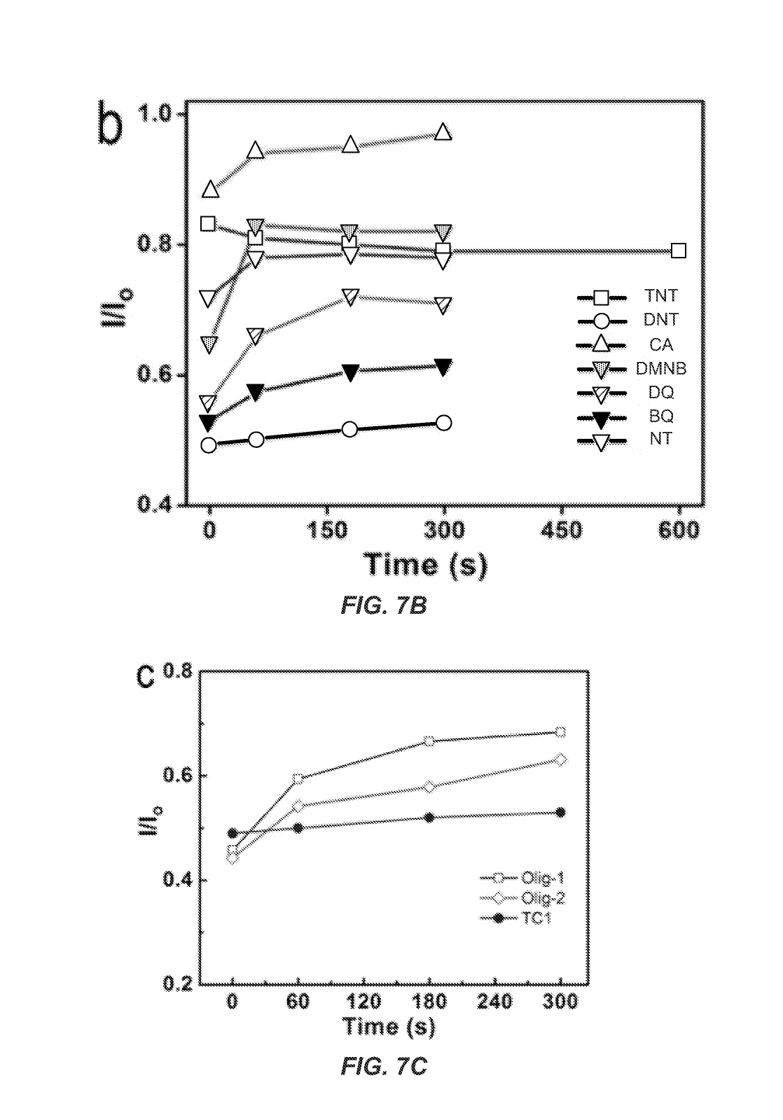

One such example was shown in FIG. 7A, where the fluorescence spectra of nanofibers of compound 1 were recorded before and after exposure to the saturated vapor of TNT (ca. 5 ppb) for one min, indicating 18% fluorescence quenching. Surprisingly, the fluorescence intensity measured over the same nanofibers kept decreasing after removing the vapor of TNT, i.e., reopening the nanofibers to clean air, as shown in FIG. 7B.

This is in contrast to the cases when exposed to other oxidizing reagent vapors, for which no such post-exposure fluorescence quenching was observed; rather the fluorescence emission was recovered to certain extents. The emission recovery is apparently due to the release of the volatile reagents from the nanofiber pores. Noticeably, the smallest extent of recovery was found for DNT, likely due to its similar size and chemical property with TNT. The post-exposure, continuous quenching observed with TNT implies some diffusion-controlled process within the porous structure, which may be constituted by the interior tubular structure of the nanofiber or the inter-fibril interstices caused by tight entangled piling of nanofibers.

In addition, considering the extended exciton migration typically expected for columnar aromatics and the fibril materials, sparse distribution of a quencher upon slow diffusion within the range of exciton migration distance also helps maximize the fluorescence quenching efficiency. The same experiments were also performed over the nanoporous nanofibers fabricated from compound 2, and demonstrated quite similar behavior as observed for the nanofibers shown FIGS. 7A and B. As such, the distinctive post-exposure fluorescence quenching observed for TNT provides a method for selectively detecting TNT from other nitro-based explosives or oxidizing reagents. The results herein, primarily relying on the dynamic characterization can open an alternative way to approach the detection selectivity. To explore whether the porosity constituted by the inter-fibril piling plays a critical role in determining the slow diffusion dynamics of TNT as observed above, the same quenching measurements as shown in FIGS. 7A and 7B were performed over the solid nanofibers fabricated from the oligomers, compounds 4 and 5. These solid nanofibers demonstrated very low fluorescence quenching upon exposure to saturated vapor of TNT, i.e., only <5% quenching was observed after 1 min of exposure. The low quenching efficiency is likely due to the poor accumulation of the low vapor of TNT. Due to this low quenching, DNT was used instead to study the post-exposure fluorescence changing as performed above over the interior porous nanofibers of compounds 1 and 2.

As shown in FIG. 7C, ca. 55% fluorescence quenching was observed for both the two nanofibers of compounds 4 and 5 upon exposure to the saturated vapor of DNT for 20 seconds (100 ppb). However, the fluorescence was considerably recovered when reopened to clean air. This is in contrast to the observation over the nanoporous nanofibers of compound 1, for which the fluorescence was recovered significantly slower, and only to a minimal extent (FIG. 7C).

These results suggest that exterior porosity constituted by the solid nanofibers can hardly afford the slow diffusion of guest molecules to enable post-exposure fluorescence quenching. Without being bound by any particular theory, this is likely due to the near micro-pore size that causes easy desorption of analytes when re-exposed (equilibrated) to clean air, thus leading to recovery of fluorescence.

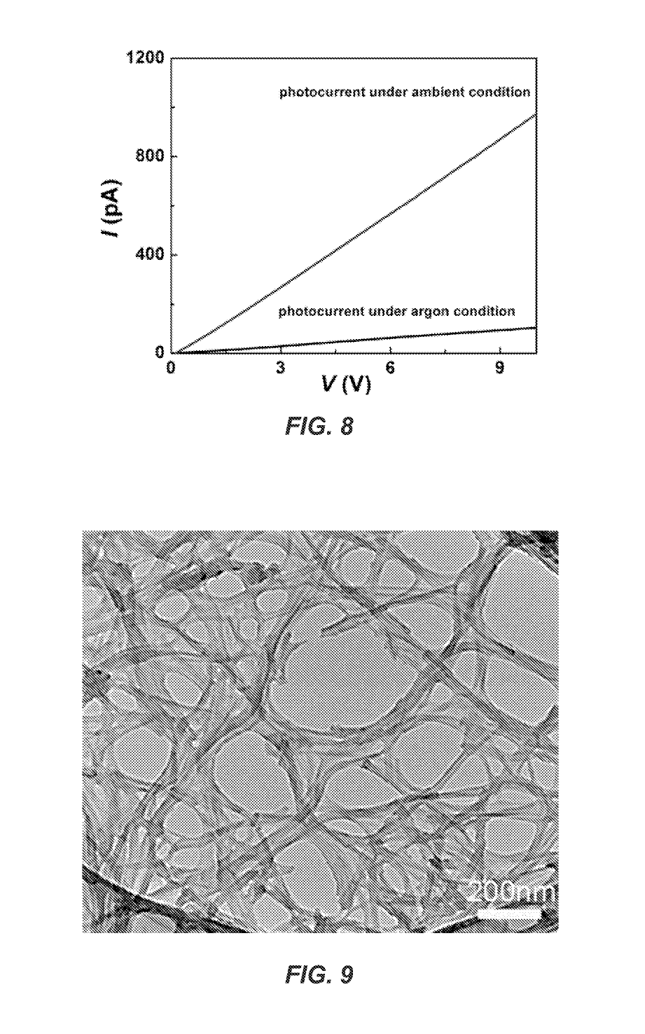

Although the inventive nanoporous nanofibers of compounds 1 and 2 have proven the feasibility of selective detection of TNT based on the post-exposure fluorescence quenching, the post-exposure fluorescence quenching can be modest and the sensor sensitivity can be relative low compared to the other fluorescence sensor systems. This is likely due to the competitive adsorption caused by the ubiquitous oxygen present inside the materials, where oxygen is believed to bind to the same site (i.e., the carbazole moiety) on the internal surface of nanofiber. The strong binding interaction between oxygen and the tetra carbazole nanofibers was implied by the enhanced photocurrent as obtained in the presence of oxygen (FIG. 8). To further increase the fluorescence sensing efficiency, reduction of the oxygen adsorption so as to accommodate more binding sites for the gas analytes can be done.

To approach this, the R groups can be modified. In one example, the tetra macromolecules of formula 1 can have functionalized R groups, e.g., compound 3. Notably, compounds 1-3 all possess the same general backbone and can be fabricated into similar tubular nanofibers (FIG. 9—TEM of nanofibers of compound 3).

Because of the introduction of a carboxyl group into the side chain linker, the HOMO energy level of the molecule is significantly lowered down to −5.6 eV, compared to that of compounds 1 and 2 (both at −4.9 eV). Decreasing the HOMO level of organic molecules represents a general strategy to improve their stability against oxygen. It is thus expected that the nanoporous nanofibers of compound 3 possess much weaker interaction with oxygen, thereby facilitating the intake of TNT. The weak interaction with oxygen of compound 3 was consistent with the negligible photocurrent as measured over the nanofibers of compound 3 in the presence of oxygen. The efficient fluorescence quenching of a spin-cast nanofibril film of compound 3 upon exposure to the vapor of TNT and DNT was previously performed.

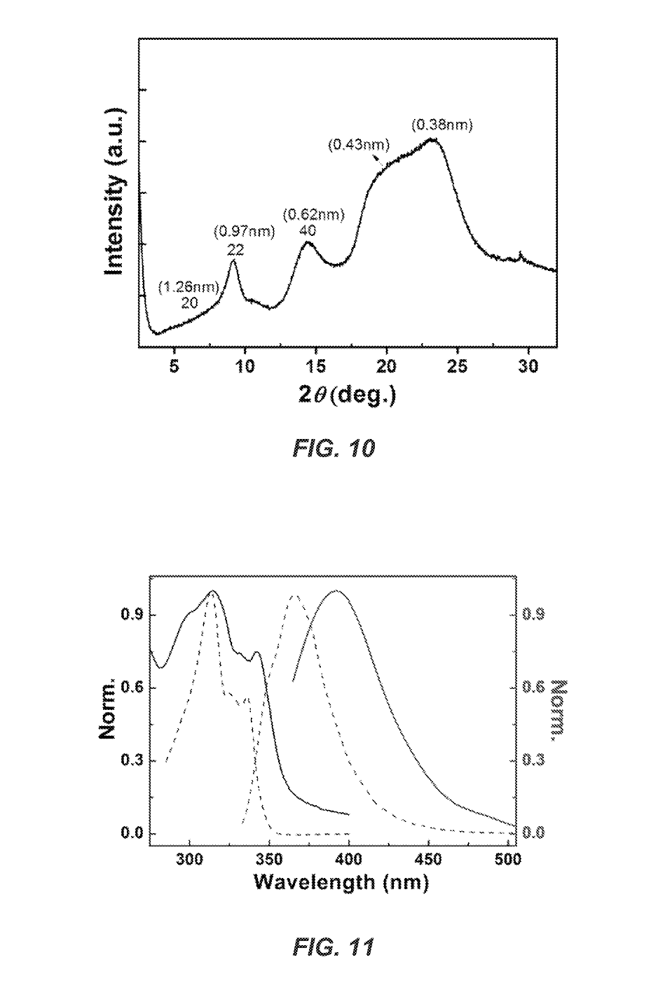

However, such previous film was found to exhibit similar fluorescence quenching behavior for various kinds of oxidizing reagents, i.e., there was a lack of selectivity for a single specific explosive target probably due to no interior pores morphology formed by this processing. The present fabricated molecules of compound 3 were formed into nanoporous nanofibers using the novel self-assembly method as employed for compounds 1 and 2. The greater intensity observed at higher-order XRD peaks (22, 40) is indicative of the tubular porous structure of the nanofiber fabricated from compound 3 (FIG. 10). Similarly to the nanofibers of compounds 1 and 2, a two-dimensional (2D) rectangular lattice with lattice parameters a and b of 2.52 nm can be deduced from the XRD data, while the formation of J-aggregation can be indicated by the optical measurements (FIG. 11).

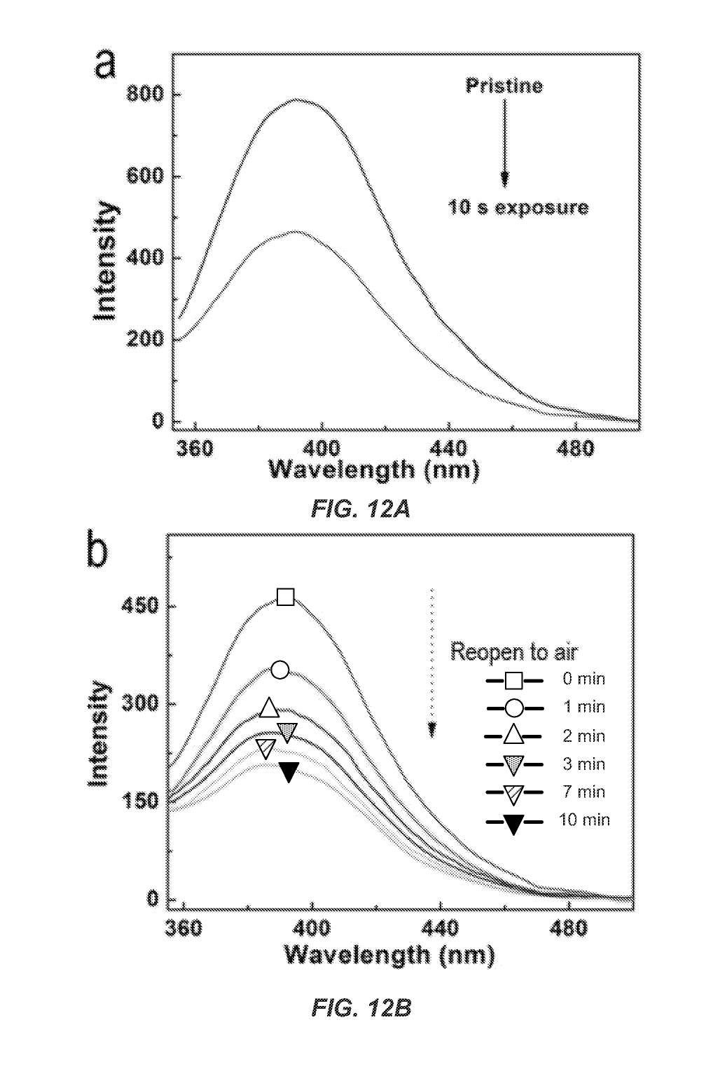

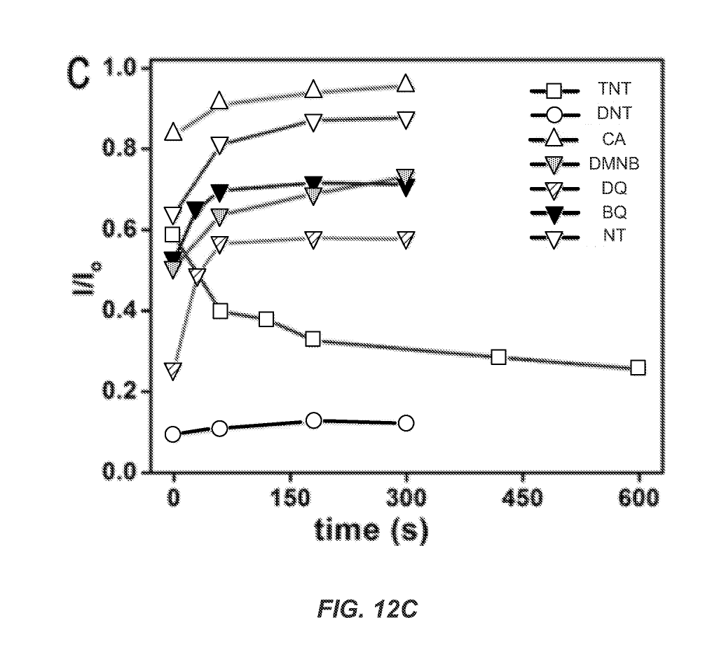

As shown in FIG. 12A, the fluorescence recorded from the nanofibers made from compound 3 was effectively quenched by 40% upon exposure to the saturated vapor of TNT for only 10 seconds. More strikingly, significant post-exposure fluorescence quenching was observed as shown in FIGS. 12B and 12C, where the fluorescence continued to decrease by another 35% within 10 min after reopening to clean air. In addition to the enhanced selectivity towards TNT as enabled by the post-exposure fluorescence quenching, the new nanofibers of compounds 3 also demonstrated enhanced sensitivity for vapor detection of this explosive, i.e., 75% fluorescence quenching was achieved upon only 10 s of initial vapor exposure. In comparison, only 20% quenching after 1 min of exposure was observed for nanofibers of compound 1 that possess the similar interior porosity as revealed by the XRD measurements.

When tested for DNT, as high as 90% fluorescence quenching was attained for the similar nanofibers of compound 3 within 10 seconds of exposure, whereas only 50% fluorescence quenching was achieved for the nanofibers of compound 1 upon 20 seconds of exposure. Clearly, a reduced interaction between the nanoporous nanofibers and oxygen can effectively improve the intake of TNT within the porosity, thereby enhancing both the sensing selectivity and sensitivity.

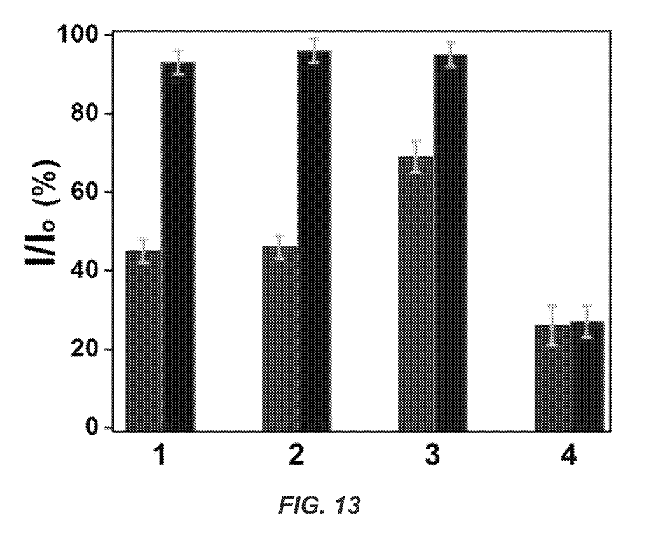

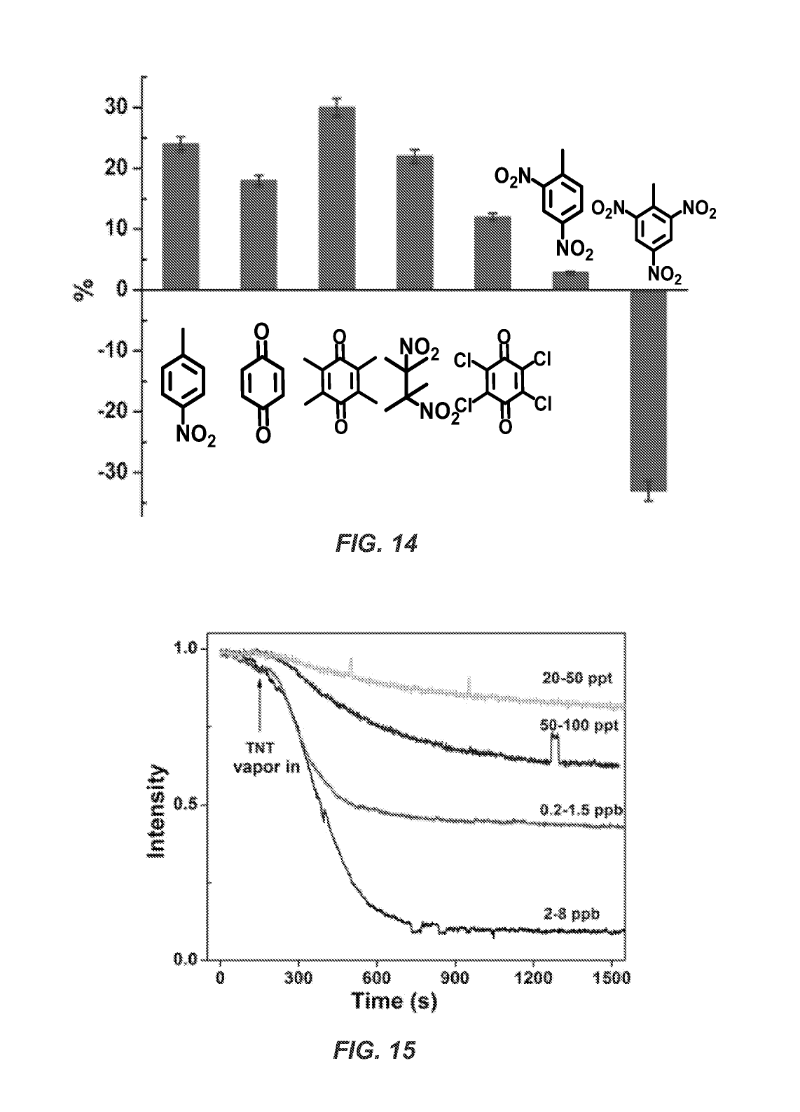

To further confirm the strong encapsulation and deep distribution of TNT within the interior pores of nanofibers, the compound 3 nanofibers after exposure were subsequently immersed in a saturated vapor of hydrazine, which has been proven effective for recovering the fluorescence of conducting polymers and organic nanomaterials after exposer to nitroaromatic explosives. Strikingly, the quenched fluorescence of the nanoporous nanofibers of compound 3 remained almost unchanged after immersing in the saturated vapor of hydrazine for 1 min followed by drying in a stream of nitrogen (FIG. 13).

In contrast, the fluorescence of the nanofibers made from compounds 4 and 5 was restored to over 90% under the same recovery conditions. Additionally, the solid nanofibers fabricated (using previous spin-cast methods) from compound 3 demonstrated a similar level of fluorescence recovery under the same experimental conditions. These results show that the performance and sensing of the organic materials is highly dependent on processing and achieving the present tubular morphology. Additionally, the unrecoverable quenching observed with the nanoporous nanofibers reflects a strong, steady-state host-guest interaction between compound 3 and TNT, which is consistent with the post-exposure quenching as observed (FIG. 12C). With the highly efficient encapsulation of vapor analytes proven above for the interior porous nanofibers, the lowest detection limit of TNT was also studied. Quantitative evaluation of the detection limit was performed at the Naval Research Lab, where a recently built vapor generator provides various levels of vapor pressures for TNT at a range of temperatures. The vapor sensing test was carried out in situ in a home-built optical chamber connected with a PMT photon detector through an optical fiber. As shown in FIG. 15, the interior porous nanofibers made from compound 3 demonstrated significant emission quenching (ca. 15%) by TNT vapor down to a few tens of ppt.

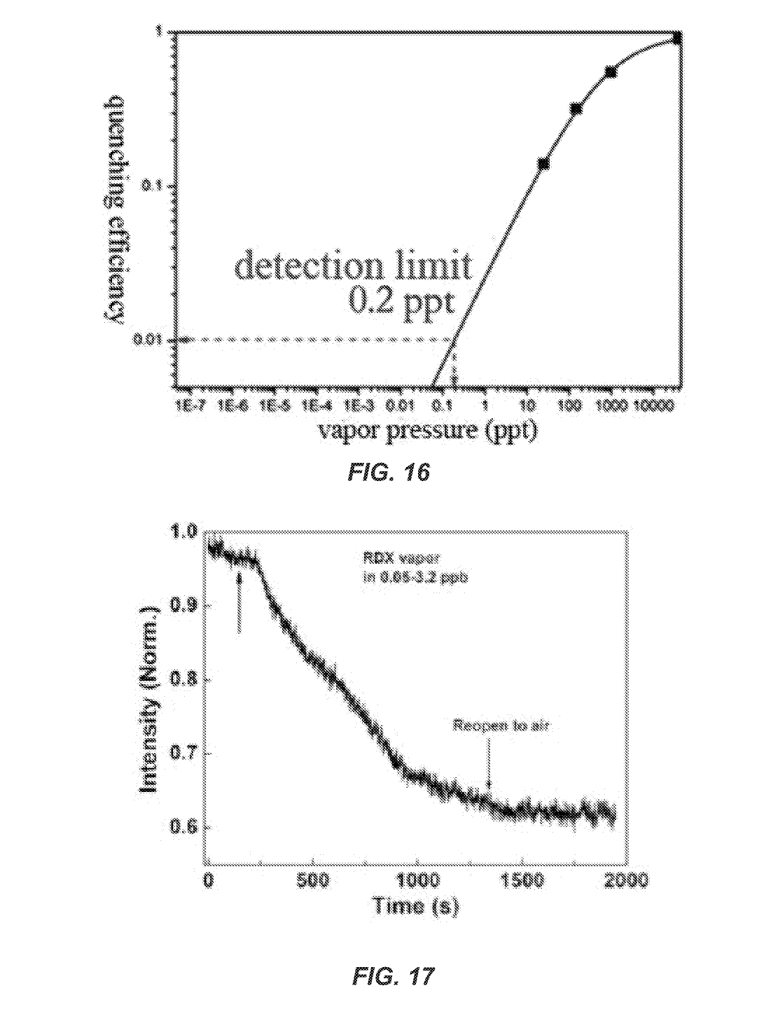

Upon increasing the vapor pressure of TNT to the range of 2-8 ppb, the quenching efficiency was increased up to ca. 90%. The total response time (about 400 s, defined from the point of introduction of TNT vapor to the turning point of quenching saturation) represents the slow vapor saturation process in the relatively large optical chamber, rather than the sensing response of the materials. To date, the obtained detection limit of few tens of ppt places the nanoporous fibers among the most sensitive materials tested under the same conditions (i.e., without elaborate optimization and integration into a instrument). Interestingly, no fluorescence recovery was observed after stopping the explosive exposure and reopening to clean air, consistent with a strong encapsulation of explosives. Further, FIG. 16 shows fluorescence quenching efficiency (1−I/I0) as a function of the vapor pressure of TNT: data (error±5%) fitted with the Langmuir equation. Notably, at 0.01 efficiency, the theoretical detection limit was 0.2 ppt.

The same tubular nanofibers were also tested for vapor sensing of RDX with the same vapor generator system. Due to the extremely low vapor pressure of RDX and its fast condensation at room temperature, the test was performed at an elevated temperature around 60° C. to maintain reliable vapor pressure at certain level. As shown in FIG. 17, about 40% emission quenching was observed upon exposure to the RDX vapor in the range of 50 ppt-3.2 ppb. This result demonstrates the first vapor detection of RDX through fluorescence quenching sensor system. The efficient sensing response is mostly due to the strong intake of RDX within the tubular void of the tetracycle, which matches the size and symmetry of RDX. Notably, for both the case of TNT and RDX no fluorescence recovery was observed after stopping the explosive exposure and reopening to clean air, consistent with a strong encapsulation of explosives. As discussed herein, the fluorescent nanofiber materials can be recovered by dissolving in chloroform followed by extraction with ethanol and water to remove explosive materials. Notably, such nanofibers reassembled from recycled material displayed the same sensing efficiency by repeat experiment.

Testing Equipment ¹H and ¹³C NMR spectra were obtained on Varian Unity 400, Unity 500, and VXR 500 spectrometers. Chemical shifts are reported in δ (ppm) relative to the residual solvent protons (CDCl₃: 7.26 for ¹H, 77.0 for ¹³C). High-resolution ESI mass spectra were recorded on a Micromass Q-Tof Ultima spectrometer. MALDI mass spectra were recorded on an Applied Biosystems Voyager-DE STR spectrometer. MALDI analysis of macrocycles was carried out using the dithranol or HABA matrix. UV-vis absorption and fluorescence spectra were measured on a LS 55 fluorometer and a PerkinElmer Lambda 25 spectrophotometer, respectively. SEM measurement was performed with a FEI NanoNova 6300 microscope, and the samples were directly drop-cast on a silica substrate. TEM was carried out on a Philips model Tecnai F20 electron microscope operating at 120 kV and in part on a JEOL 2100 Cryo TEM at 200 kV in the Frederick Seitz Materials Research Laboratory Central Facilities, University of Illinois. Powder The X-ray diffraction was carried out with a Philips X'Pert XRD instrument. Electrical current measurements of the nanofibers were carried out using a two-probe method on a Signatone S-1160 Probe Station combined with an Agilent 4156C Precision Semiconductor Parameter Analyzer for high-resolution current measurement. The micro-gap electrodes were fabricated by photolithography on a silicon wafer covered with a 300-nm thick SiO₂ dielectric layer. The gold electrode pair used here was 14 μm in width and 5 μm in gap, and fully covered with nanofibers via drop-casting. A tungsten lamp (Quartzline, 21V, 150 W) was used as the white light source for photocurrent generation, and the light is guided into the probe station through a glass optical fiber, followed by focusing on the sample through the objective lens. Vapor generator was home-made by Navy Research Lab.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A fluorescence-based sensor comprising:
a nanofiber mass of nanofibers having tubular morphology, the nanofibers comprising carbazole-cornered, arylene-ethynylene tetracyclic macromolecules of formula I: 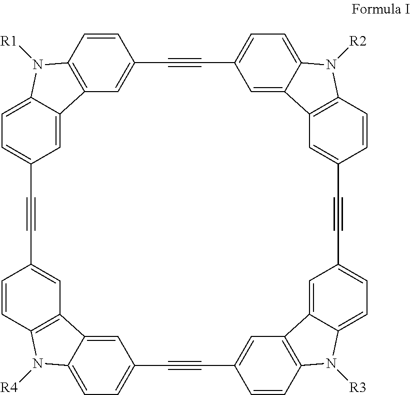
wherein R1-R4 are alkyl-containing groups that facilitate cofacial stacking to form the tubular morphology, and wherein at least some of the macromolecules are cofacially stacked; and
a fluorescence detector;
wherein fluorescence of the nanofibers decreases upon contact with a nitro-containing compound.

2. The sensor of claim 1, wherein the sensor is contained in a housing that contains the nanofiber mass and fluorescence detector and is portable.

3. The sensor of claim 1, wherein R1-R4 are individually selected from C3 to C18 alkyl chains.

4. The sensor of claim 1, wherein R1-R4 are C14 alkyl chains.

5. The sensor of claim 1, wherein R1-R4 are individually selected from C3 to C18 functionalized alkyl chains.

6. The sensor of claim 1, wherein R1-R4 are each

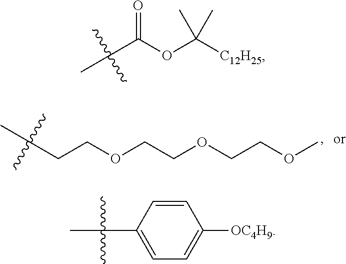

7. The sensor of claim 1, wherein the nanofibers have a diameter of about 10 nm to about 100 nm and wherein the nanofiber mass is a film.

8. The sensor of claim 1, wherein the sensor detects trinitrotoluene in a concentration as low as 0.2 ppt.

9. The sensor of claim 1, wherein the sensor is selective of trinitrotoluene over other oxidizing organic compounds by measuring a different post-exposure fluorescence change profile affected by trinitrotoluene compared to the other oxidizing organic compounds.

10. The sensor of claim 1, wherein the nitro-containing compound is an explosive.

11. The sensor of claim 1, wherein the nitro-containing compound is trinitrotoluene.

12. A method of manufacturing the nanofibers of claim 1, comprising:
solvating the carbazole-cornered, arylene-ethynylene tetracyclic macromolecules in a first organic solvent forming a macromolecule solution;
admixing the macromolecule solution with a second organic solvent forming a binary solvent system;
cooling the binary solvent system to a temperature of at least 4° C. for a period of at least 6 days thereby forming the nanofibers having tubular morphology.

13. The method of claim 12, wherein the first organic solvent is a halogen-containing solvent and the second organic solvent is an alcohol.

14. The method of claim 12, wherein the macromolecule solution has a macromolecule concentration ranging from 0.01 mM to 10 mM.

15. A method of detecting explosives, comprising:
exposing nanofibers having a tubular morphology to a target sample, the nanofibers comprising carbazole-cornered, arylene-ethynylene tetracyclic macromolecules of formula I: 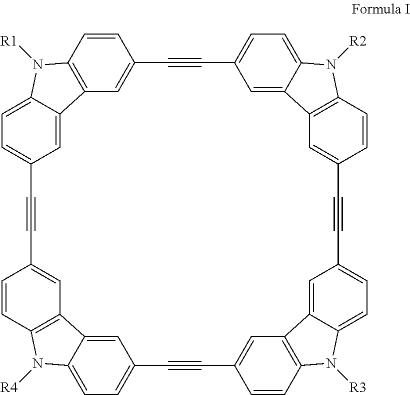
wherein R1-R4 are alkyl-containing groups and wherein the macromolecules are cofacially stacked; and
measuring fluorescence responses of the nanofibers.

16. The method of claim 15, wherein the target sample is trinitrotoluene (TNT).

17. The method of claim 15, further comprising exposing the nanofibers to an ambient gas and measuring a post-exposure fluorescence.

18. The method of claim 17, wherein the method selectively detects TNT over other nitro-containing compounds based on the post-exposure fluorescence.

19. The method of claim 17, wherein the ambient gas is a member selected from the group consisting of air, a noble gas, an inert gas, and mixtures thereof.

20. The method of claim 15, further comprising displaying an explosives indicator based on the fluorescence responses, wherein the explosives indicator is a quantitative measurement or a qualitative measurement.

21. The method of claim 15, wherein the fluorescence responses are statistically significant at target sample concentrations of about 0.2 ppt and greater.

22. The method of claim 15, further comprising recycling the nanofibers by dissolving the nanofibers in a first organic solvent and extracting the nanofibers with a second organic solvent.

23. The method of claim 22, wherein the first organic solvent is a halogen-containing solvent and the second organic solvent is an aqueous alcohol mixture.
